(19) United States
(12) Patent Application Publication
(10) Pub. No.: US 2026/0108679 A1
(43) Pub. Date: Apr. 23, 2026

(54) DISPENSER HAVING FORCE-MODULATING COMPONENTS

(71) Applicant: Merck Sharp & Dohme LLC, Rahway, NJ (US)

(72) Inventors: Igor Inga, Wayne, NJ (US); Mukund P. Patankar, Parsippany, NJ (US)

(73) Assignee: Merck Sharp & Dohme LLC, Rahway, NJ (US)

(21) Appl. No.: 19/359,857

(22) Filed: Oct. 16, 2025

Related U.S. Application Data
(60) Provisional application No. 63/710,051, filed on Oct. 22, 2024.

(51) Int. Cl. A61M 5/315

(52) U.S. Cl. CPC A61M 5/31581; A61M 5/31505; A61M 5/31573

(57) ABSTRACT

A dispenser includes a thumb press having a first surface and a plurality of teeth on an opposing end of the first surface, a drive nut having at least one tab engageable with the plurality of teeth, a combo nut coupleable to the drive nut and being capable of rotation, and a lead screw coupled to the combo nut and being at least partially disposed within the combo nut, the lead screw being configured and arranged to axially translate with respect to the combo nut when the thumb press is linearly actuated.

### CROSS-REFERENCE TO RELATED APPLICATIONS

This application claims the benefit of U.S. Provisional Patent Application Serial No. 63/710,051 filed October 22, 2024, the entire contents of which are incorporated by reference herein

### FIELD OF THE INVENTION

The present disclosure relates generally to dispensers, syringes and injectors. More specifically, the present disclosure relates to dispensers, needle-based syringes and injectors that have force-modulating components.

### BACKGROUND OF THE INVENTION

A typical syringe known to those in the art includes a plunger rod and stopper combination that leads to at least two shortcomings. First, movement of the plunger stopper cannot be accurately controlled, and therefore control of the volume of the delivered dose is compromised. This may also pose a concern where less than the full dose is to be delivered (e.g., delivery of a partial dose from a syringe barrel). This is particularly true for highly potent drugs and substances that require precise dosage control. Secondly, the force required to deliver a medicament of high viscosity can be uncomfortably high for the user administering the medicament. This is because the stopper pressure required to deliver a substance from a syringe barrel is proportional to the viscosity of the substance.

Thus, there exists a need for devices that improve upon and advance methods of safely using injectors and syringes, such as pre-filled syringes, to ensure superior control of the delivered dose, and to reduce the discomfort of delivering high viscosity medicaments.

### SUMMARY OF THE INVENTION

In some examples, a dispenser includes a thumb press having a first surface and a plurality of teeth on an opposing end of the first surface, a drive nut having at least one tab engageable with the plurality of teeth, a combo nut coupleable to the drive nut and being capable of rotation, and a lead screw coupled to the combo nut and being at least partially disposed within the combo nut, the lead screw being configured and arranged to axially translate with respect to the combo nut when the thumb press is linearly actuated.

In some examples, a method of injecting a substance includes providing a dispenser including a thumb press having a first surface and a plurality of teeth on an opposing end of the first surface, a drive nut having at least one tab engageable with the plurality of teeth, a combo nut coupleable to the drive nut and being capable of rotation, and a lead screw coupled to the combo nut and being at least partially disposed within the combo nut, the lead screw being configured and arranged to axially translate when the thumb press is linearly actuated, coupling the dispenser to a pre-filled syringe having a substance, and injecting the substance from the pre-filled syringe by actuating the thumb press.

### BRIEF DESCRIPTION OF THE DRAWINGS

Various embodiments of the presently disclosed syringes are disclosed herein with reference to the drawings, wherein:

FIG. 1 is a schematic front view of a pre-filled syringe;

FIGS. 2A-B are schematic perspective and cross-sectional views of a dispenser according to one example of the present disclosure;

FIG. 3 is an exploded, schematic perspective view of a dispenser and pre-filled syringe;

FIG. 4 is a perspective view of an embodiment of a combo nut assembly;

FIG. 5 is a perspective view of an embodiment of a combo nut assembly in relation to a barrel, thumb press and a return cap;

FIG. 6 is a partial cross-sectional view of an embodiment of a combo nut assembly in relation to a barrel, thumb press and a return cap;

FIG. 7 is another partial cross-sectional view of an embodiment of a combo nut assembly in relation to a barrel, thumb press and a return cap;

FIG. 8 is a perspective view showing the assembly of a pre-filled syringe with the dispenser;

FIG. 9 is a cross-sectional view of the assembled dispenser and pre-filled syringe; and

FIGS. 10A-D are schematic perspective views showing details of certain components of the dispenser.

Various embodiments are described below with reference to the appended drawings. It is to be appreciated that these drawings depict only some embodiments of the disclosure and are therefore not to be considered limiting of its scope.

### DETAILED DESCRIPTION OF THE INVENTION

Despite the various improvements that have been made to injectors and syringes, such as pre-filled syringes, conventional methods suffer from some shortcomings as discussed above.

Therefore, there is a need for further improvements to the devices and methods used to deliver medication and reduce discomfort to the user for high viscosity medicaments. Among other advantages, the present disclosure may address one or more of these needs.

As used herein, the term “proximal,” when used in connection with a component of a syringe or injector, refers to the end of the component closest to the user’s hands when holding the device; whereas the term “distal,” when used in connection with a component of a syringe or injector, refers to the end of the component closest to the needle insertion site during use. Likewise, the terms “trailing” and “leading” are to be taken as relative to the operator’s fingers (e.g., physician) of the syringe or injector. “Trailing” is to be understood as relatively close to the operator’s fingers, and “leading” is to be understood as relatively farther away from the operator’s fingers. Moreover, as used herein, the terms “medicament,” “medication,” and “drug” are used generically interchangeably and it will be understood that the ampoules described herein may be used to store, deliver, or administer vaccines, biologics, therapeutics, medicaments, topical ointments, and the like.

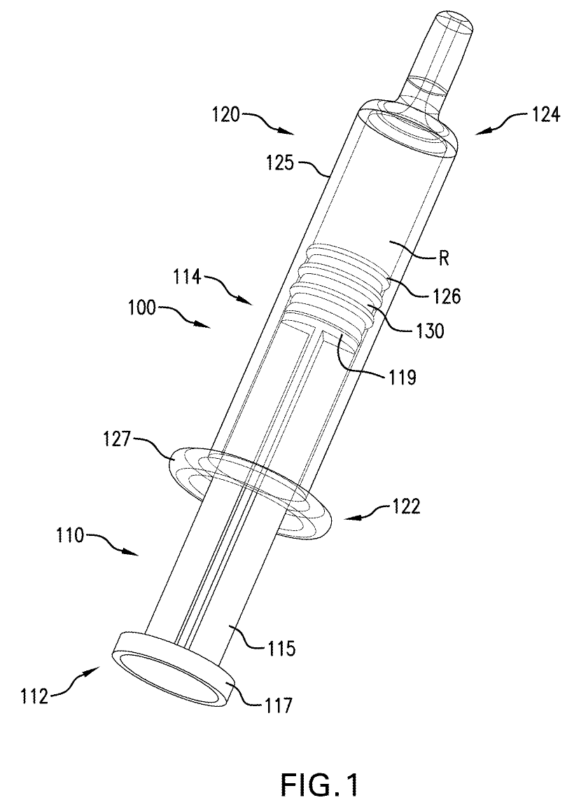

Reference is now made to FIG. 1, which shows an exemplary prefilled-syringe 100 contained within a needle safety device known in the art. It will be understood that though a needle within a safety device is shown, the disclosure is not thus limited. For example, though a pre-filled syringe with a staked needle is shown, it will be understood that the principles disclosed herein are equally applicable to other types of injectors (e.g., syringes with removable needles, auto-injectors, or on-body (wearable) injectors having needles, etc.). Pre-filled syringe 100 generally comprises two main portions, a plunger rod assembly 110 and a barrel 120. Plunger rod assembly 110 generally extends between a proximal end 112 and a distal end 114, and generally comprises an elongated piston 115 extending between a plunger flange (or press) 117 and a stopper 130.

A cylindrical barrel 120 extends between proximal end 122 and distal end 124 and comprises a body 125 defining a lumen 126 for accepting a portion of plunger rod assembly 110. Body 125 further comprises a barrel flange 127 adjacent proximal end 122 and defines a reservoir “R” that holds a medicament, drug, saline, or other substance for injecting into a patient’s body. An internally threaded stopper 130 is disposed inside lumen 126 of body 125. In one embodiment, stopper 130 is made of an elastomeric material such as natural rubber, synthetic rubber, thermoplastic elastomers, or combinations thereof, and comprises an opening to receive and mate with a portion of plunger rod assembly 110 by advancing a portion of the plunger rod assembly inside the barrel lumen 126 and rotating at least one of coupler 119 and stopper 130 relative to the other. Though not shown, a syringe may optionally include a cap 135 disposed over a needle coupled to the distal end of the barrel 120. Once the cap removed, the user may pierce the patient’s skin with the needle, then push on plunger flange 117 to drive the plunger to deliver a medicament through needle into the patient’s body.

Prefilled-syringe 100 is prone to some of the shortcoming described above with respect to precise control of a delivered dose, especially partial doses, and discomfort to the user for delivery of high viscosity medicaments. Thus, there exists a need for a dispenser or syringe assembly, whereby a full translation of an actuator results in movement of the plunger stopper along only a portion of the length of the syringe barrel, and whereby repeated distal/proximal translation(s) of the actuator creates repeated limited and precise distal movements of the plunger stopper. The features described herein may be attached to a standard syringe plunger stopper to impart the attributes of precise distal movement of the plunger stopper along the length of a syringe barrel while reducing the force exerted by the user to deliver a quantity of a medicament.

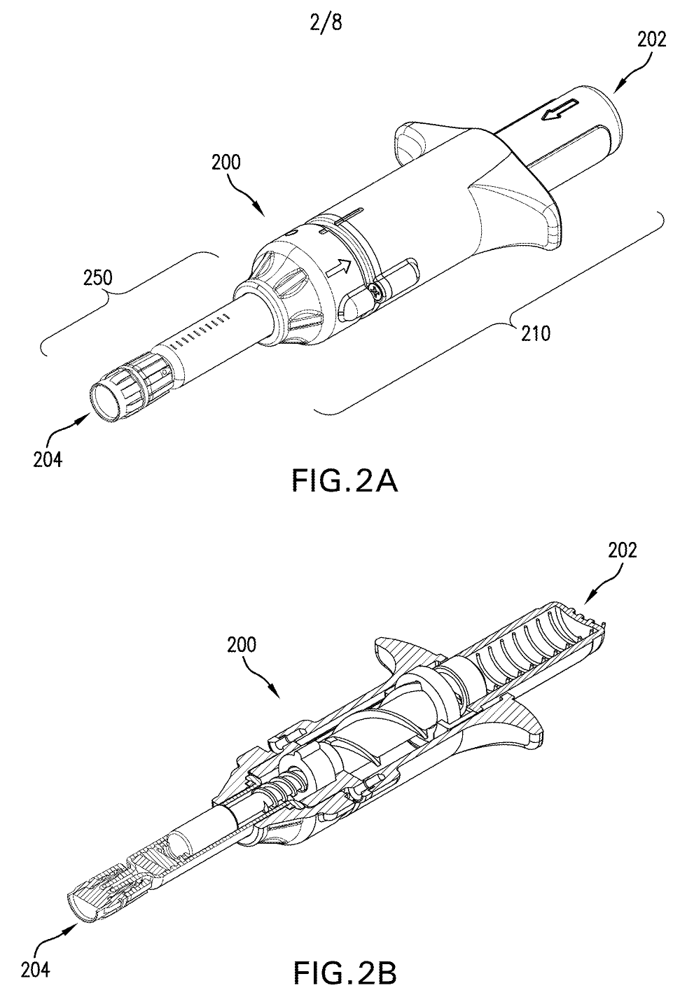

Instead of solely using translation to deliver medicament, a dispenser according to the present disclosure may rely on conversion from translational to rotational forces, and from rotational forces back to axial translation, to achieve these goals. FIGS. 2A-B are schematic perspective views of a dispenser 200 that extends between a proximal end 202 and a distal end 204. In FIG. 2B, a cutaway view is shown so that the interior of the dispenser can be appreciated. In this example, dispenser 200 includes two basic subassemblies: a proximal subassembly 210 disposed closer to the proximal end 202, and a distal subassembly 250 including a standard pre-filled syringe, the distal subassembly 250 being disposed closer to distal end 204. The two subassemblies 210, 250 are complementary, and cooperate to resolve some of the shortcomings of prior art devices (e.g., difficulty in injecting high viscosity substances through a syringe).

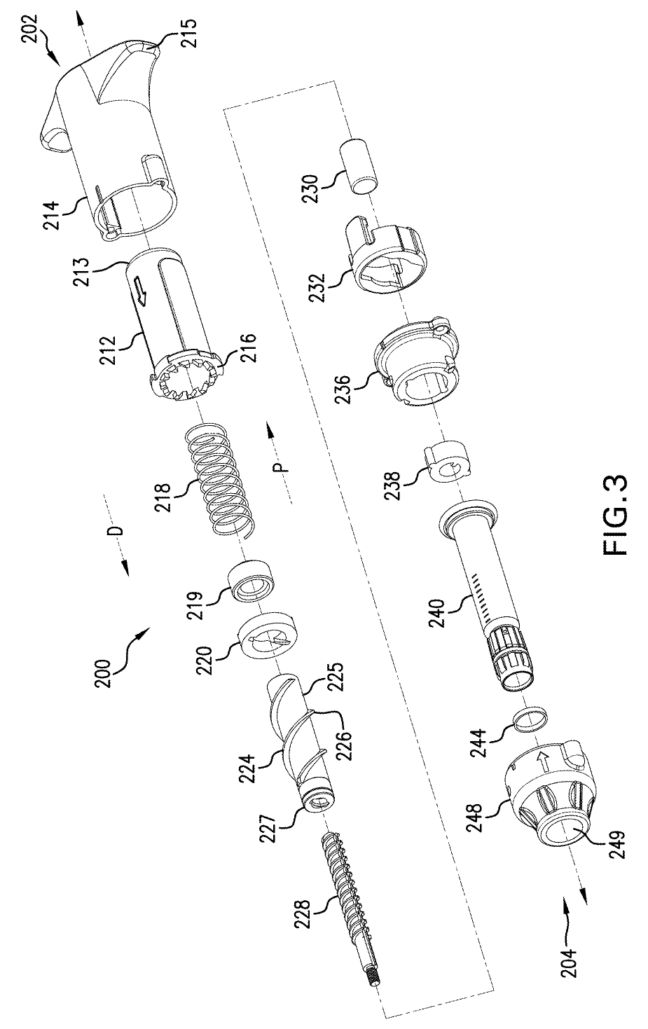

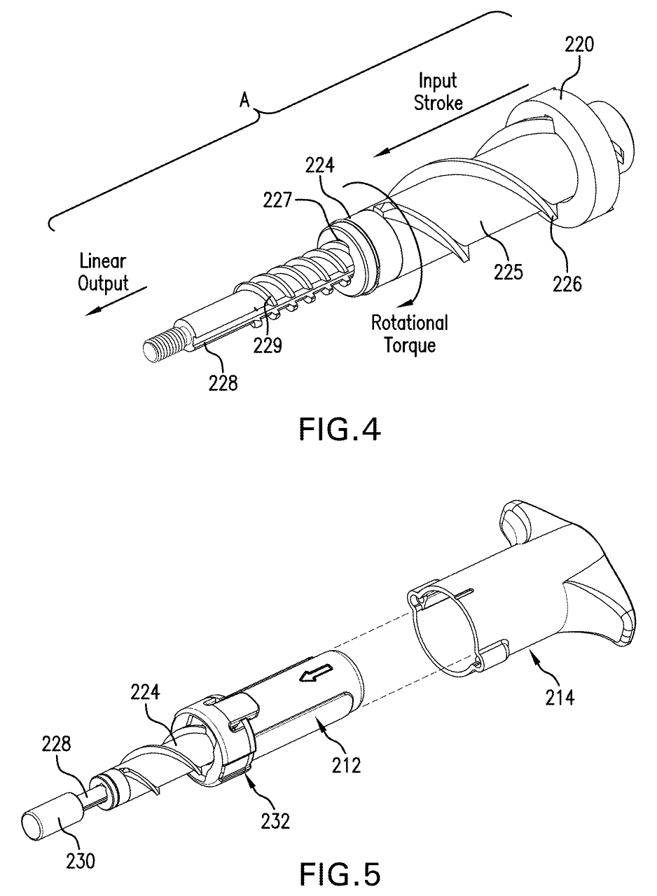

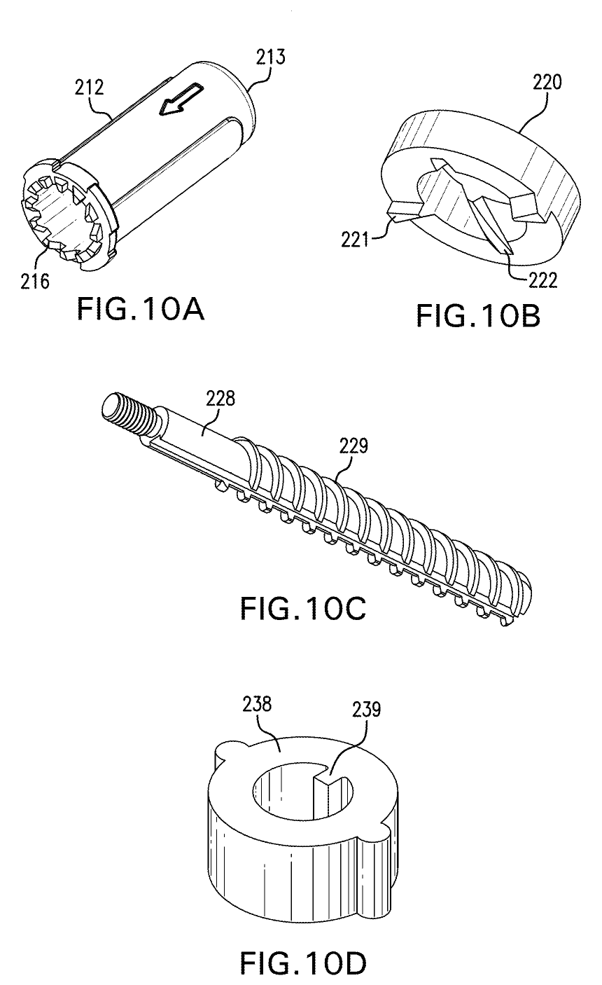

FIG. 3 illustrates an exploded view of the dispenser 200 and FIGS. 4-7 illustrate some of those components as they cooperate with one another. Beginning at the proximal end 202, proximal subassembly 210 generally includes a cylindrical thumb press 212 at least partially disposed within a barrel 214. The initial load to the dispenser 200 may be applied by the user to the thumb press 212. Thumb press 212 may include a flat or smooth upper surface 213 where the user’s thumb, or other digit, will be placed. In this example, thumb press 212 may be at least partially disposed within barrel 214 and translatable relative thereto, and barrel 214 may include two opposing flanges 215 that allow the user’s fingers (e.g., index finger and middle finger) to rest onto as a third finger (e.g., the thumb) pushes on upper surface 213. Thumb press 212 may include a series of annular teeth 216 disposed on an end opposite upper surface 213 (See, also, FIG. 10A, which shows a detailed view of an exemplary thumb press with the annular teeth 216 exposed).

Thumb press 212 may at least partially house an internal mechanism which converts the linear load exerted by the user (e.g., the user’s thumb) to a rotational torque. In some examples, this conversion utilizes a “Yankee screw” concept. Specifically, a user may repeatedly push on thumb press 212 in the direction of arrow “D” to deliver a medicament, and the thumb press 212 may return to its original position after each push. To properly return the thumb press 212 to its initial position, a spring 218 may be disposed within thumb press 212 and configured to urge thumb press 212 toward proximal end 202 when the force is released from upper surface 213 of thumb press 212. Spring 218 may be seated within thumb press 212 and fixed on the other side via spring support 219. In some examples, spring support 219 may also be at least partially (or fully) disposed within thumb press 212. Spring 218 may allow the subassembly to reload by returning the thumb press 212 in the proximal direction in the direction of arrow “P” back to its original location after each press.

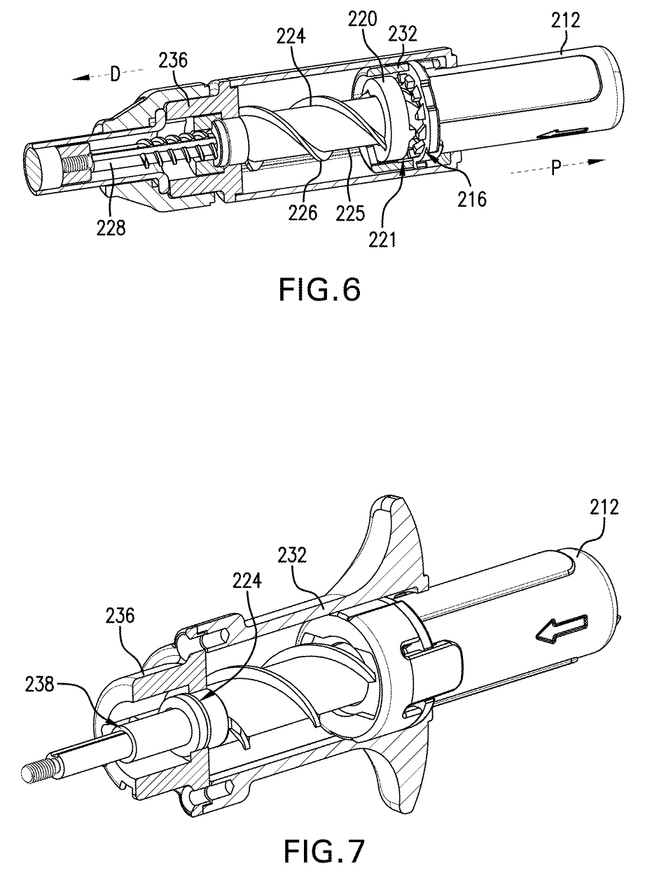

In conjunction with thumb press 212, drive nut 220, combo nut 224 and lead screw 228 may represent the main features of a combo nut assembly “A” that takes an input axial stroke, converts it to a rotational torque and results in an axial or linear output (FIGS. 4-7). A drive nut 220 may be mechanically engaged with annular teeth 216 of thumb press 212. In this example, drive nut 220 includes at least one tab 221 mechanically engageable with annular teeth 216 (FIG. 6). Details of drive nut 220 are better shown in FIG. 10B. In some examples, drive nut 220 includes multiple tabs 221 (e.g., two tabs) that can be unidirectionally driven by annular teeth 216. This mechanism may allow teeth 216 to rotate drive nut 220 when the user pushes the thumb press 212 distally towards the syringe barrel in the distal direction shown by arrow “D.” In this manner, drive nut 220 may be unidirectionally rotationally configured. The user may apply any number of strokes to thumb press 212 to create additional rotation of drive nut 220. Drive nut 220 may further include an inner recess 222 for receiving a portion of combo nut 224 (FIG. 10B).

Coupled to drive nut 220 is combo nut 224, which may have a substantially cylindrical body 225 and an external helical thread 226 engageable with inner recess 222 of drive nut 220. In some examples, helical thread 226 may be an external thread with a first pitch of 20 mm to 50 mm or from 30 mm to 40 mm. In the example shown, external helical thread 226 may load against a support plate of a barrel cap 236, which causes the combo nut 224 to rotate without axial translation. Combo nut 224 may also include an inner helical recess 227 that receives portions (e.g., threads) of lead screw 228. Lead screw 228 may be configured to translate with respect to combo nut 224 (e.g., lead screw 228 may translate in or out of combo nut 224). In some examples, the turning of combo nut 224 causes lead screw 228 to translate as threads 229 of lead screw 228 travel within the inner recess 227 of combo nut 224 (See, FIG. 10C for a more detailed view of lead screw 228). In some examples, the lead screw threads 229 may have a second pitch of from 2 mm to 8 mm, or from 4 mm to 6 mm. In some examples, the second pitch is finer than the first pitch of the combo nut and the difference between the first pitch and the second pitch modulates the amount of force (i.e., the axial load) required to axially translate a stopper over a predetermined distance. A cylindrical plunger 230 may be coupled to the distal end of lead screw 228 and may translate therewith. Taken together, the components of combo nut assembly “A” are intended to convert an axial movement of thumb press 212 to rotational torque in combo nut 224 and back to an axial movement or linear output in lead screw 228 and/or plunger 230. Axial movement of the plunger 230 may in turn urge a stopper of a pre-filled syringe to expel a substance out of the syringe barrel through its lumen. This conversion may provide a mechanical advantage (e.g., an 8:1, 7:1, 6:1 or 5:1 mechanical advantage). For example, an initial force of 6 lbs. by the user’s thumb on thumb press 212 may result in an output force of 40.4 lbs. In some examples, an input force of 4 lbf. (17.8 N) may be added to 1 lbf (4.4 N) of spring force so that the user experiences 5 lbf (or 22.2 N). In some examples, the dispenser may require a maximum of 4 input strokes to deliver the contents of a pre-filled syringe.

To properly couple combo nut assembly “A” to the remainder of the dispenser, a return cap 232 is provided (best shown in FIG. 5), which is coupled to and optionally fitted over thumb press 212. Return cap 232 may be fixedly attached to thumb press 212 and may serve to prevent rotation of the thumb press 212 within barrel 214. In other words, return cap 232 may be incapable of rotating with respect to thumb press 212, barrel 214 or both. A barrel cap 236 and key ring 238 may be coupled to the distal end of combo nut 224. As previously noted, barrel cap 236 may form a surface against which combo nut 224 rests (FIG. 7). Barrel cap 236 may also support key ring 238 which includes an inner-projecting rib 239 to lock lead screw 228 and prevents it from rotating (FIG. 10D).

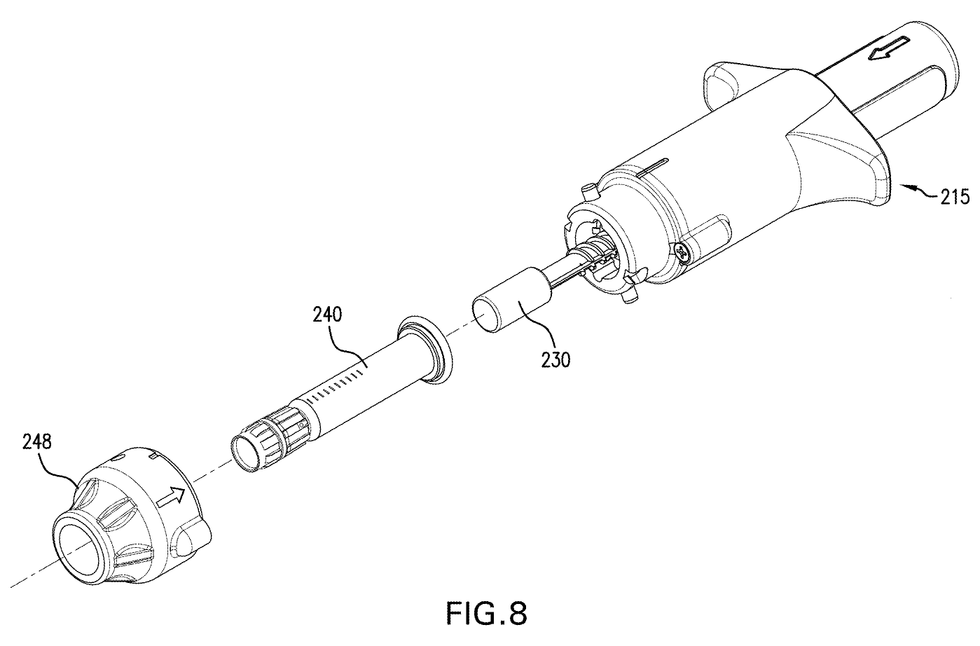

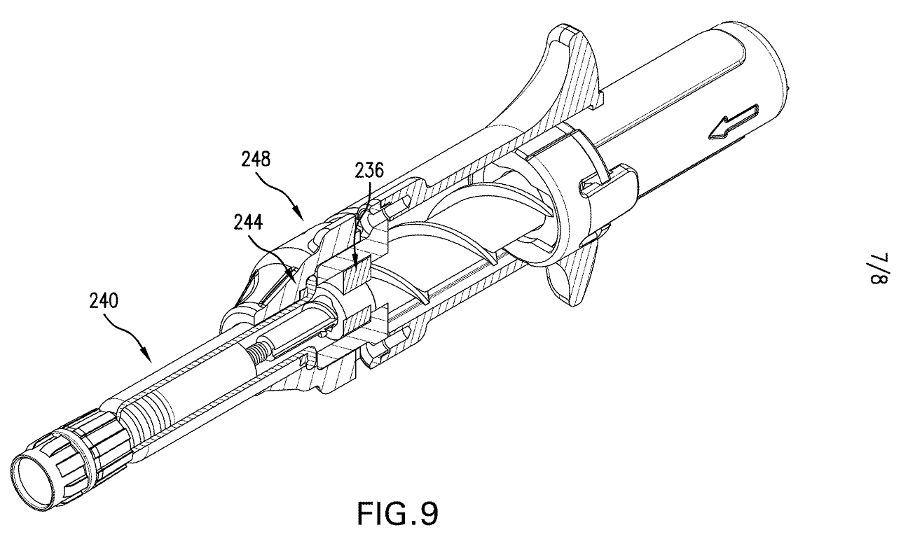

A distal cap 248 is configured to engage with barrel cap 236 to close the distal end of the dispenser 200. Distal cap 248 may have a lumen 249 sized to receive a portion of pre-filled syringe while retaining a portion of the syringe (e.g., a barrel flange). As shown in FIG. 8, a pre-filled syringe 240 may be disposed between the plunger 230 and distal cap 248 and concentrically aligned with lead screw 228, combo nut 224 and thumb press 212. In some examples, pre-filled syringe 240 is a 1-mL pre-filled syringe. Of course, other sizes and shapes of the pre-filled syringes are possible. Optionally, an O-ring 244 is disposed between the distal end of pre-filled syringe 240 and distal cap 248. In some examples, pre-filled syringe 240 is placed into distal cap 248 and is attached to the distal cap by twisting it (e.g., a 90-degree twist). This engagement of pre-filled syringe 240 with distal cap 248 may result in a 25% compression of O-ring 244 to secure the syringe in place during use. FIG. 9 illustrates the dispenser and pre-filled syringe after assembly.

In use, the dispensers, syringes or injectors of the present disclosure may be used to deliver high viscosity fluids (e.g., fluids with a viscosity of from about 0 centipoise to about 40 centipoise, of from about 10 centipoise to about 30 centipoise, or from about 15 centipoise to about 25 centipoise, when measured with a viscometer (e.g., BROOKFIELD DV-II+Pro Viscometer) at 20 °C at a standard concentration). For example, high viscosity fluids may require applying significant linear forces to a plunger rod of a conventional device to expel fluid from a traditional syringe. One advantage of the present disclosure is that a syringe according to one or more of these embodiments may be used to inject high viscosity fluids with a reduced force on the thumb press. The injection may also be properly metered for more precise dosing. For example, each full travel of the thumb press may deliver a known fraction of the substance from a syringe barrel (e.g., 1/10, 1/8, 1/6, 1/5, 1/4, 1/3 or 1/2 of the contents). The reduced force required to use the syringe may also allow high viscosity fluids to be easily delivered by magnifying the output to provide a higher force on the plunger rod to expel the highly-viscous fluid.

Assemblies similar to those described herein may be manufactured as a standalone device (e.g., a dispenser as shown) or by retrofitting existing pre-filled syringe assemblies by threading into the plunger stopper. In some examples, the subassemblies may include various components that allow the user to apply a linear axial load and distal motion similar to that of an existing plunger rod design. The application of the load operates similar to traditional plunger rods where the user applies force with their thumb to the backend of the assembly. The internal mechanism comprising a helical thread converts this linear load to a rotational torque, while a second, internal mechanism within the system, comprised of a second helical thread, then converts the rotational torque back to a linear load to provide additional mechanical advantage for the user. Loading may be repeated several times to exercise the plunger rod along the total length of the syringe barrel to deliver the substance contained inside syringe. In some examples, repeated loading may be tied to a metering system that correlates to a viscosity of the medicament and the user may be instructed to actuate the thumb press a predetermined number of cycles based on the dose to be delivered. Repetition of loading will be driven by the spring mechanism within the proximal subassembly to return the thumb press to an extended position. Thus, the concepts described increase the applied stopper force applied by the user through the mechanism over a predetermined travel of the thumb press. Stated another way, the forces required for urging a stopper over the same distance may be decreased through this mechanism. Of course, modifications are possible to vary the desired force outputs by changing the properties of the combo nut, the lead screw, the first pitch, the second pitch, the ratio of the first to second pitch, etc.

It is to be understood that the embodiments described herein are merely illustrative of the principles and applications of the present disclosure. For example, the volume and material for the various components of the syringe may be varied. Moreover, certain components or steps of a method of using the device are optional, and the disclosure contemplates various configurations and combinations of the steps disclosed herein. Additionally, as used herein, the term “coupleable” refers to two or more components that cooperate, join or engage one another. It will be understood that where two or more components are said to be “coupled” or “coupleable” that they may also be unitarily or integrally formed. For example, certain components of proximal subassembly 210 may be integrally or unitarily formed with one another, or certain components of distal subassembly 250 may be integrally or unitarily formed with one another. Additionally, or alternatively, components of proximal and distal subassemblies 210, 250 may be unitarily formed with one another. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A dispenser, comprising:
a thumb press having a first surface and a plurality of teeth on an opposing end of the first surface;
a drive nut having at least one tab engageable with the plurality of teeth;
a combo nut coupleable to the drive nut and being capable of rotation; and
a lead screw coupled to the combo nut and being at least partially disposed within the combo nut, the lead screw being configured and arranged to axially translate with respect to the combo nut when the thumb press is linearly actuated.

2. The dispenser of claim 1, wherein the combo nut has a helical external thread having a first pitch.

3. The dispenser of claim 1, wherein the drive nut is disposed on an outer circumference of the combo nut, the drive nut defining an inner recess that receives the helical external thread of the combo nut.

4. The dispenser of claim 1, wherein the lead screw has a helical external thread having a second pitch.

5. The dispenser of claim 4, wherein the second pitch is finer than the first pitch.

6. The dispenser of claim 2, wherein the combo nut has a cylindrical body that defines an inner helical recess for receiving the helical external thread of the lead screw.

7. The dispenser of claim 1, wherein the combo nut is unidirectionally rotatable.

8. The dispenser of claim 1, further comprising a spring disposed within the thumb press.

9. The dispenser of claim 1, further comprising a cylindrical plunger coupled to the lead screw.

10. The dispenser of claim 1, further comprising a return cap disposed adjacent a distal edge of the thumb press and being coupleable to at least one of the thumb press and the drive nut.

11. The dispenser of claim 1, further comprising a barrel cap and a distal cap disposed over a portion of the lead screw and being configured to retain a pre-filled syringe in concentric alignment with the lead screw.

12. The dispenser of claim 11, wherein the lead screw is rotationally fixed with respect to the barrel cap and incapable of rotation.

13. The dispenser of claim 1, further comprising an O-ring disposed within the distal cap.

14. The dispenser of claim 1, further comprising a barrel having two opposing flanges, the thumb press being at least partially disposed within the barrel and translatable relative thereto.

15. The dispenser of claim 13, wherein the combo nut is fixed with respect to the barrel and incapable of axial translation.

16. An injection device comprising:
the dispenser of claim 1; and
a pre-filled syringe having a barrel defining a reservoir and a stopper.

17. A method of injecting a substance comprising:
providing a dispenser including a thumb press having a first surface and a plurality of teeth on an opposing end of the first surface, a drive nut having at least one tab engageable with the plurality of teeth, a combo nut coupleable to the drive nut and being capable of rotation, and a lead screw coupled to the combo nut and being at least partially disposed within the combo nut, the lead screw being configured and arranged to axially translate with respect to the combo nut when the thumb press is linearly actuated;
coupling the dispenser to a pre-filled syringe having a substance; and
injecting the substance from the pre-filled syringe by actuating the thumb press.

18. The method of claim 17, wherein injecting the substance by actuating the thumb press comprises linearly actuating the thumb press to rotate the combo nut.

19. The method of claim 17, further comprising the step of linearly actuating the lead screw.

20. The method of claim 17, further comprising pressing the thumb press more than once.
